(19) United States
(12) Patent Application Publication
(10) Pub. No.: US 2012/0059032 A1
(43) Pub. Date: Mar. 8, 2012

(54) 1,2,3,4-TETRAHYDROISOQUINOLINE DERIVATIVES EFFECTIVE AS ANTIGLIOMA AGENTS, METHODS OF MAKING, AND THEIR USE

(75) Inventors: Renukadevi Patil, Memphis, TN (US); Shivaputra A. Patil, Memphis, TN (US); Duane D. Miller, Germantown, TN (US); Charles Ryan Yates, Collierville, TN (US); Eldon E. Geisert, Germantown, TN (US)

(73) Assignee: University of Tennessee Research Foundation, Memphis, TN (US)

(21) Appl. No.: 13/042,273

(22) Filed: Mar. 7, 2011

Related U.S. Application Data
(63) Continuation-in-part of application No. 12/859,234, filed on Aug. 18, 2010, now abandoned.
(60) Provisional application No. 61/234,629, filed on Aug. 18, 2009.

(51) Int. Cl. A61K 31/472; A61P 35/00; A61K 31/4725; C07D 217/20; C07D 401/10

(52) U.S. Cl. 514/307; 546/149; 546/148

(57) ABSTRACT

Disclosed are tetrahydroisoquinoline-derivative compounds effective for killing cancer cells, shrinking tumor size, and treating cancer.

### CROSS-REFERENCE TO RELATED APPLICATIONS

This application is a continuation-in-part of U.S. patent application Ser. No. 12/859,234, filed on Aug. 18, 2010, which claimed the benefit of priority of U.S. Provisional Patent Application No. 61/234,629, filed on Aug. 18, 2009.

### FIELD OF USE

The present invention relates to compounds for use as chemotherapeutic agents. More specifically, the invention relates to derivatives of 1,2,3,4-tetrahydroisoquinoline compounds for use as anti-tumor agents and in the treatment of cancer.

### BACKGROUND OF THE INVENTION

Significant progress has been made in the treatment of certain cancers, and the prognosis for individuals with some types of cancers, such as prostate, breast, thyroid, and skin cancer (i.e., certain melanomas) is generally good for at least 4 out of 5 patients. According to the United States National Institutes of Health, however, the prognosis for individuals with cancers such as those of the brain (27.3% survival), lung (13.4% survival), liver (6.0% survival), and pancreas (3.2% survival) has been poor, based upon statistics compiled from 1983 to 1990. According to the American Cancer Society, almost 22,000 people in the United States alone develop brain and nervous system cancers each year, and about 13,070 people die from these cancers. About 42 percent of all brain tumors are gliomas. Grade IV astrocytoma (glioblastoma multiforme, or GBM), for example, is considered by many to be the most malignant brain tumor. The average length of survival for people with GBM is 12 to 14 months, and common symptoms of GBM are seizures, nausea and vomiting, headaches that become progressively worse, a declining ability to move certain parts of the body, and weakness or numbness in the face or arm. GBM rapidly affects the quality of the patient's life, and it may affect individuals of any age.

Discovering and developing effective therapeutic agents for difficult-to-treat cancers is important both for the benefit of individuals who are diagnosed with those cancers, as well as for individuals who may have other difficult-to-treat forms of cancer. For example, researchers at the University of Minnesota's Masonic Cancer Center have discovered a genetic link between two types of cancers for which effective treatment has been achieved in only a small fraction of the individuals diagnosed with those cancers—glioblastoma and leukemia. Agents that are found to effectively treat one form of these difficult cancers may therefore provide excellent lead compounds for testing as therapeutic agents for other cancers which are known to be more refractory to treatment. What are needed are agents that effectively treat cancers and provide increased life expectancy and life quality for thousands of individuals who are diagnosed with cancer each year.

### SUMMARY OF THE INVENTION

The present invention relates to compounds for the treatment of cancer, these compounds comprising 1,2,3,4-tetrahydroisoquinoline derivatives having structures as in Formula (I)

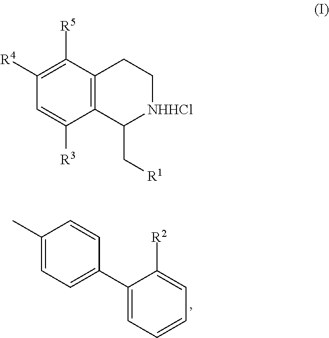

where R¹ is

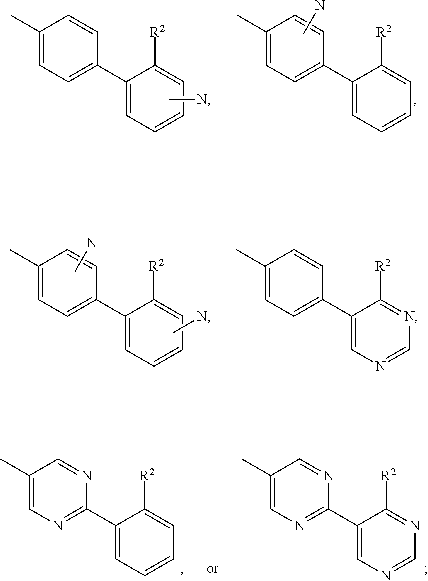

R² is —OCH₃, —CF₃, —NHSO₂CH₃, —NHCOCH₃, —SO₂CH₃, —N(CH₃)₂, —CN, —NO₂, —NH₂, —CO₂CH₃, —OCF₃, —CH₃, —F, —Br, or —I;

R³ is —OCH₃; and

R⁴ and R⁵ are —H or —OCH₃, wherein when R⁴ is —H, R⁵ is —OCH₃ and when R⁴ is —OCH₃, R⁵ is —H.

One aspect of the invention comprises compounds of Formula (I)

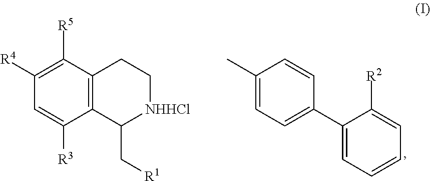

where R¹ is

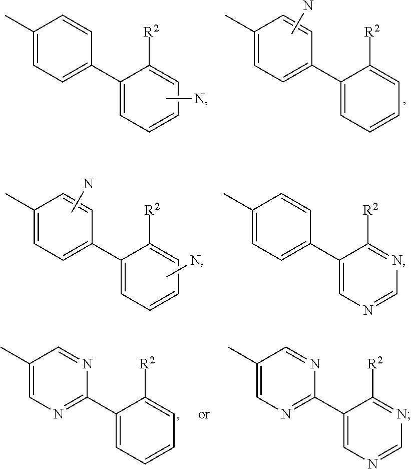

and R² is —OCH₃, these compounds being particularly effective for the selective destruction of tumor cells and the treatment of cancer.

The invention also provides methods for killing cancer cells, shrinking tumor size, and treating cancer, those methods comprising administering to a patient one or more compounds of Formula (I) as described above. In some aspects, the cancer cells are glioblastoma cells. In some aspects, the compound may be a compound of Formula I wherein R¹ is

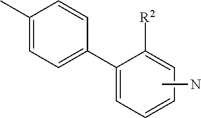

### and R² is —OCH₃.

### BRIEF DESCRIPTION OF THE DRAWINGS

FIG. 1 (Scheme 1) illustrates the synthesis of 6,8-dimethoxy-1-(2′-methoxybiphenyl-4-ylmethyl)-1,2,3,4-tetrahydroisoquinoline hydrochloride 9a and 6,8-dimethoxy-1-[4-(4-methoxypyridin-3-yl)benzyl]-2-methyl-1,2,3,4-tetrahydroisoquinoline dihydrochloride 9b.

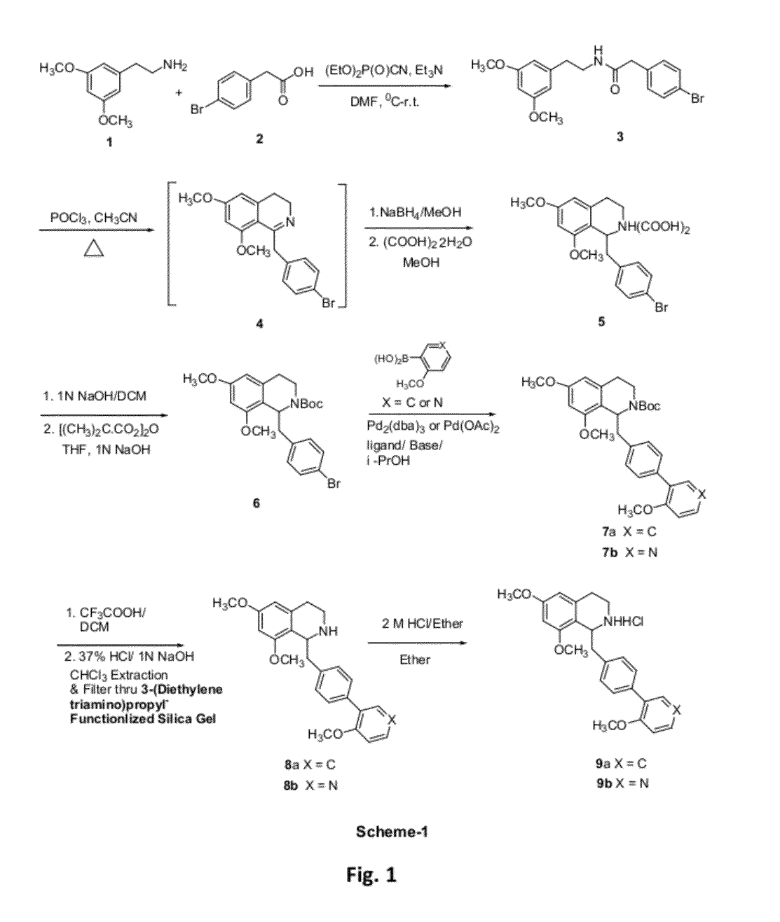

FIG. 2 (Scheme 2) is a synthetic scheme illustrating the preparation of hydrochloride salts 12a and 13a. Individual isomers were resolved by (R, R) WHELK-01 chiral HPLC column (Regis Technologies, Morton Grove, Ill.), enabling the inventors to obtain both enantiomers in nearly 100% optical purity with an 85:15 ratio of Hexane and isopropanol. After resolution, both the isomers, isomer-I&II (10a & 11a) were treated with trifluoroacetic acid in dichloromethane followed by reaction with 2M HCl in ether to afford corresponding hydrochloride salts 12a and 13a, respectively (Scheme-2).

FIG. 3 (Scheme 3) is a synthetic scheme for the synthesis of compounds 17a, 17b, 18a, and 18b.

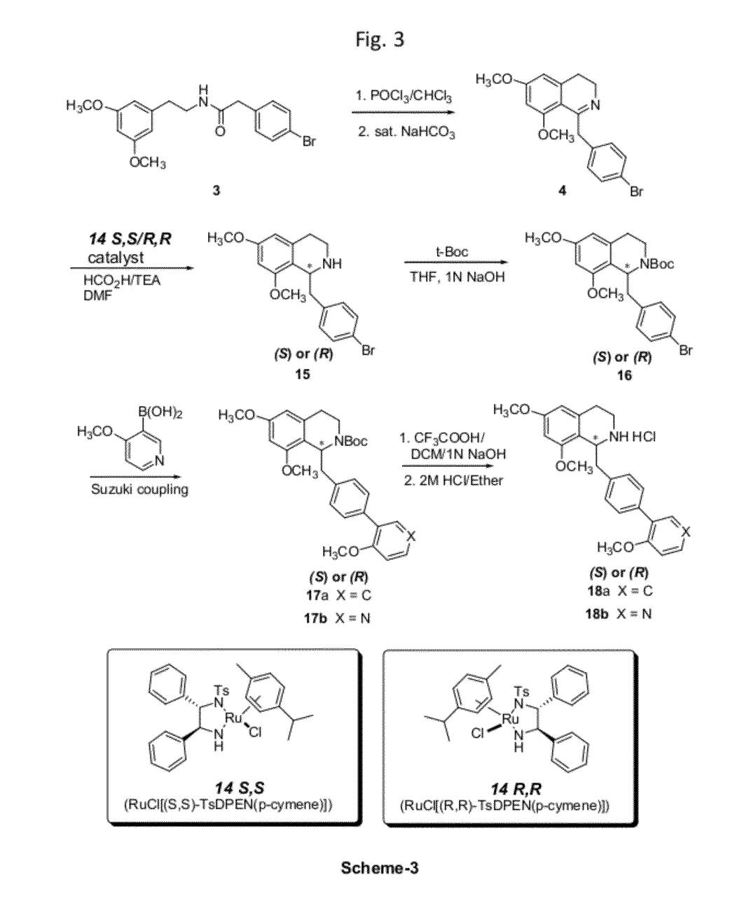

FIG. 4 is a series of graphs illustrating the results of chiral separation of compound 7a using LC/DAD. UV spectrum of isomer I (10a) is shown in panel A and isomer II (11a) in panel B. Separation of racemic mixture (7a) is shown in panel C. X-axis=nm; Y-axis=units absorbance (AU).

FIG. 5 is a series of dose response curves testing the effects of 7a/13a on three different cell types. The first curve tests the effects of 7a/13a on primary cultures of normal rat astrocytes. The calculated EC50 is 17.8 μM. The second curve is for C6 rat glioma cells and the EC50 of 0.06 μM indicates that these cells are an order of magnitude more sensitive to the compound. The third curve is from cultures of human U251 glioma. The EC50 of 0.91 indicates that EDL-355 is killing glioma at a sub-μM concentration. X-axis=EDL-355 Log₁₀ concentration in μM; Y-axis=percent survival.

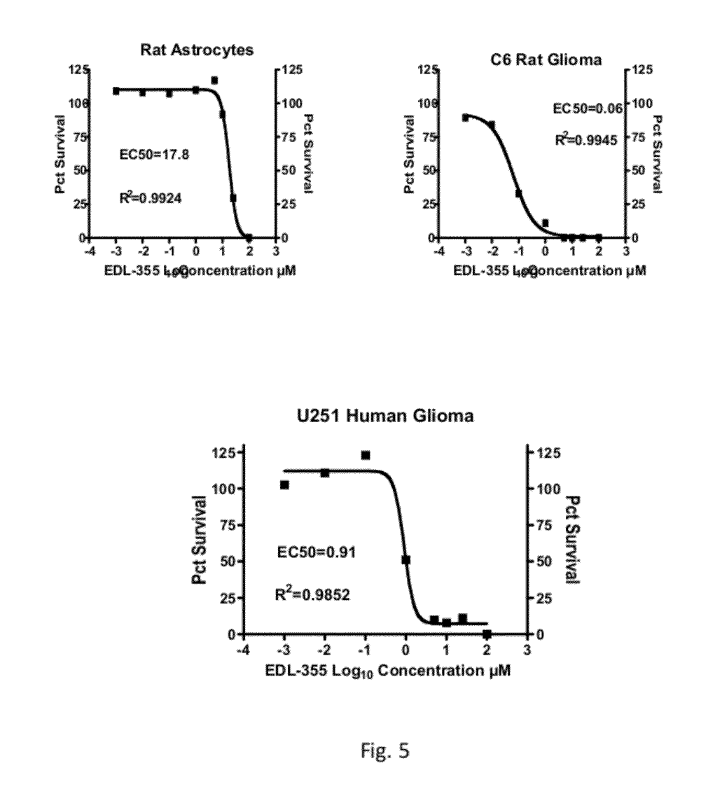

FIG. 6 is a graph of the results of the use of a side pocket model to test the effects of 7a/13a on Human U251 glioma in the nude mouse. Lines 1-5 are numbered starting from the top. The upper line (1) shows the growth of tumor in control mice (n=10) receiving carrier solution only. Line number 4 represents mice receiving a closely related compound EDL-291 (n=15). Line number 3 represents mice receiving EDL-291 and temezolomide (TMZ) (n=9). Line number 2 represents animals receiving TMZ alone (n=9). Line number 5 represents animals receiving EDL-355. A decrease in tumor size was noted with EDL-355 (n=5) that was significantly different from any of the other four treatment groups (p<0.01, student t test). X-axis=number of days; Y-axis=normalized tumor volume in mm³.

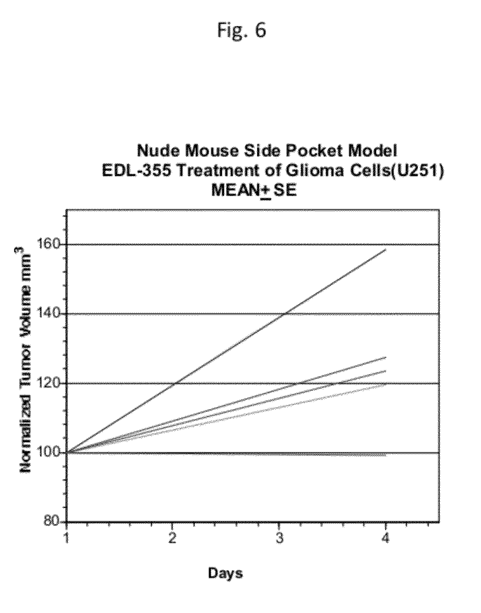

### DETAILED DESCRIPTION

The inventors have synthesized 1,2,3,4-tetrahydroisoquinoline derivatives and have shown that these compounds are highly effective agents for the selective destruction of cancer cells. For example, compounds of the invention have demonstrated significant efficacy in selectively destroying glioma cells, while effective doses for destruction of tumor cells appear to have no noticeable detrimental effects on normal cells. Compounds of the invention have been shown to destroy tumor cells and shrink or eradicate tumors in vivo, and are therefore effective agents for the treatment of cancer. One class of cancers for which these compounds show efficacy is brain cancers, including glioblastoma. Compounds of the invention may also be effective agents for other cancers, as well.

Compounds of the invention may be described by Formula (I)

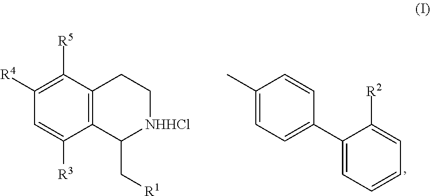

where R¹ is

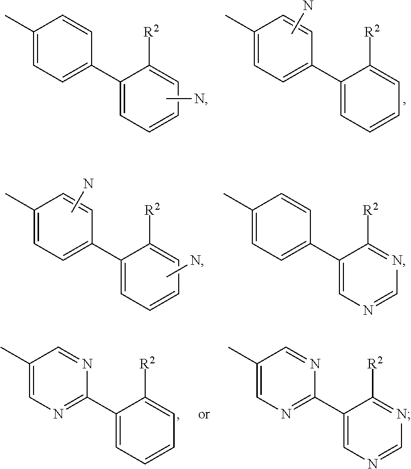

R² is —OCH₃, —CF₃, —NHSO₂CH₃, —NHCOCH₃, —SO₂CH₃, —N(CH₃)₂, —CN, —NO₂, —NH₂, —CO₂CH₃, —OCF₃, —CH₃, —F, —Br, or —I;

R³ is —OCH₃; and

R⁴ and R⁵ are —H or —OCH₃, wherein when R⁴ is —H, R⁵ is —OCH₃ and when R⁴ is —OCH₃, R⁵ is —H.

The inventors have also developed methods for the treatment of cancer and/or for decreasing tumor size, killing cancer cells, etc., comprising administering to a patient with cancer a therapeutically effective dose of one or more compounds disclosed herein. One or more compounds of the invention may be provided in conjunction with other therapeutic agents for the treatment of cancer, if desired by the treating physician, and/or compounds of the invention may be administered in conjunction with one or more nutritional compositions containing, for example, vitamins, minerals, enzymes, co-factors, and/or nutritional compounds/compositions. Compounds of the invention may also be provided in conjunction with anti-inflammatory agents, agents for the treatment of pain, etc. For example, one or more compounds of the present invention may be provided in a therapeutic regimen that includes the administration of an agent such as aspirin (acetylsalicylic acid), one or more compounds of the invention may be provided in a therapeutic regimen that includes administration of vitamin E (e.g., alpha-tocopherol, delta-tocopherol, gamma-tocopherol, alpha-tocotrienol, delta-tocotrienol, gamma-tocotrienol, or combinations thereof).

Compounds of the invention may be administered orally, subcutaneously, intraperitoneally, intravenously, topically, or by other means known to those of skill in the medical arts. Where appropriate, such compounds may be administered by an appropriate health care professional or they may, where appropriate, be self-administered by the patient.

The synthesis of 6,8-dimethoxy-1-(2′-methoxybiphenyl-4-ylmethyl)-1,2,3,4-tetrahydroisoquinoline hydrochloride 9a and 6,8-dimethoxy-1-[4-(4-methoxypyridin-3-yl)benzyl]-2-methyl-1,2,3,4-tetrahydroisoquinoline dihydrochloride 9b is shown in scheme-1. The acid (2) was reacted with 2-(3,5-dimethoxyphenyl)ethylamine (1) in the presence of diethylcyanophosphonate and triethyl amine in anhydrous DMF to obtain the amide derivative (3). The amide 3 was cyclized by Bischler-Napieralski reaction using POCl₃ in anhydrous acetonitrile followed by reduction with NaBH₄ to provide free amine, which was then treated with oxalic acid in methanol to form the more stable oxalyl salt (5). The oxalyl salt 5 was converted to free amine using 1N NaOH in dichloromethane prior to use. The free amine was allowed to react with di-tert-butyldicarbonate to yield N-substituted-1,2,3,4-tetrahydroisoquinoline (6). Compound 6 was coupled with 2-methoxyphenyl/4-methoxypyridine-3-boronic acids using Suzuki reaction conditions to produce compounds 7a/7b. Finally, compounds 7a and 7b were treated with trifluoroacetic acid in dichloromethane under stirring conditions, followed by acid/base extractions to provide free amines 8a/8b, which were then treated with 2M HCl in ether to provide hydrochloride salts 9a and 9b in good yields.

### Enantioselective Synthesis of THI-Analogs

The synthesis of chiral cyclic amines such as THI derivatives by enantioselective asymmetric imine reduction using Noyori reagents as recently emerged as powerful and versatile method in drug discovery and development. Enantiomers of chiral compounds may demonstrate significant differences in their PK/PD profiles. In order to establish the enantiopharmacological profiles of 9a/9b, the inventors performed the enantioselective synthesis of (R)- and (S)- of 9a/9b by catalytic asymmetric transfer of hydrogenation of imines using 2-propanol and/or formic acid as a hydrogen source. Ruthenium catalysts (Noyori reagents) 14SS and 14RR may be used for the stereospecific synthesis of 9a/9b. These catalysts are prepared in situ in the reaction with conversion of imine to amine. The amide 3 is subjected to Bischler-Napieralski cyclization using POCl₃. The crude iminium salt is subsequently converted to free imine 4 by means of neutralization using saturated sodium bicarbonate. The catalytic system is prepared freshly every time by reacting the dimeric dichloro(p-cymene)ruthenium(II) with 1,2-(R, R)—N-tosyl-1,2-diphenethylenediamine (for S-isomer) or 1,2-(S,S)—N-tosyl-1,2-diphenethylenediamine (for R-isomer) in presence of triethyl amine in DMF at 90° C. The warm solution is added to dihydroisoquinoline derivative 4 in DMF and the reaction mixture is cooled to 0° C. A mixture of formic acid and triethyl amine (5:2) is added to the cooled mixture. The formed tetrahydroisoquinoline 15 (S or R) is purified using flash chromatography. Protection of tetrahydroisoquinoline with t-Boc is achieved using NaOH in THF. The absolute configuration is determined by X-ray. Compounds 17a & 17b (S or R) and final hydrochloride salts 18a & 18b (S or R) are synthesized using similar experimental conditions as mentioned for compounds 7a & 7b and 9a & 9b, respectively.

### 2-(4-Bromophenyl)-N-[2-(3,5-dimethoxyphenyl)ethyl]acetamide 3

To a stirred solution of 4-bromophenyl acetic acid 2 (0.500 g, 2.33 mmol) and 2-(3,5-dimethoxyphenyl)ethylamine 1 (0.463 g, 2.59 mmol) in anhydrous DMF (10 mL) was added Et₃N (0.588 g, 5.81 mmol) followed by diethyl cyanophosphonate (0.417 g, 2.55 mmol) at 0° C. The reaction mixture was stirred at room temperature for 22 h and then it was poured into 100 mL of water. The precipitated solid was collected, washed with water (2×50 mL) and air dried overnight. The crude product was recrystallized from ethyl acetate-hexane to afford 3 (0.820 g, 93%) mp: 86-88° C.; ¹H NMR (d₆-DMSO): δ 8.10 (t, J=5.1 Hz, 1H, —NH), 7.46 (d, J=8.4 Hz, 2H, ArH), 7.16 (d, J=8.1 Hz, 2H, ArH), 6.33 (s, 3H, ArH), 3.70 (s, 6H, 2*OCH₃), 3.36 (s, 2H, —CH₂), 3.27 (q, J=7.2, 6.9 Hz, 2H, —CH₂), 2.63 (t, J=7.2 Hz, 2H, —CH₂); MS (ESI): m/z 400 [M+Na]⁺. Anal. Calcd (C₁₈H₂₀BrNO₃) C, H, N.

### 1-(4-Bromobenzyl)-6,8-dimethoxy-1,2,3,4-tetrahydroisoquinoline oxalate 5

Phosphorus oxychloride (49.494 g, 322.8 mmol) was added to a stirred solution of 3 (4.07 g, 10.76 mmol) in anhydrous acetonitrile (100 mL) and refluxed for 6 h. The reaction mixture was concentrated under reduced pressure. Methanol (2×25 mL) was added to the reaction mixture and the solvent evaporated under reduced pressure. Sodium borohydride (6.11 g, 161.4 mmol) was added to the residue in methanol (50 mL) and the reaction mixture was stirred overnight at room temperature. The solvent was removed under reduced pressure, and the resulting oily mass was dissolved in CHCl₃, washed with 1N NaOH followed by water and dried over Na₂SO₄. The solvent was removed under reduced pressure and the residue was dissolved in CHCl₃. A solution of oxalic acid dihydrate (2.71 g, 21.52 mmol) in methanol was added to the above solution with stirring at room temperature, followed by the addition of ether. The mixture was stirred overnight at the same temperature, and the solid filtered, washed with ether and air dried to provide 5 (3.9 g, 80%) as white solid; mp: 168-70° C.; ¹H NMR (d₆-DMSO): δ 8.45-8.20 (bs, 1H, NH), 7.53 (d, J=8.1 Hz, 2H, ArH), 7.21 (d, J=8.4 Hz, 2H, ArH), 6.46 (s, 1H, ArH), 6.42 (s, 1H, ArH), 4.70 (t, J=6.6 Hz, 1H, —CH), 3.76 (s, 3H, —OCH₃), 3.68 (s, 3H, —OCH₃), 3.42-3.35 (m, 2H, —CH₂), 3.11-2.93 (m, 4H, 2*-CH₂); MS (ESI): m/z 362 [M-(COOH)₂+H]⁺. Anal. Calcd (C₂₀H₂₂BrNO₆) C, H, N.

### 1-(4-Bromobenzyl)-6,8-dimethoxy-3,4-dihydro-1H-isoquinoline-2-carboxylic acid tert-butyl ester 6

To a suspension of 5 (3.8 g, 8.40 mmol) in DCM (250 mL) was added 1N NaOH (150 mL) and the mixture was stirred at room temperature for 3 h. The two layers were separated and the aqueous layer was extracted with DCM (2×50 mL) and dried over Na₂SO₄. The solvents were removed under reduced pressure to yield free amine, as yellow oil. A solution of di-tert-butyl dicarbonate (2.75 g, 12.6 mmol) in THF (30 mL) was added to a stirred solution of the oil in THF (50 mL) and aqueous 1N NaOH (40 mL) at 0° C. The reaction mixture was stirred over night at room temperature and the solvents were concentrated under reduced pressure. The residue was diluted with water and extracted with DCM. The organic layer was dried over Na₂SO₄ and the solvent was evaporated under reduced pressure. Crude residue was purified by flash column chromatography using acetone-hexane (20:80 to 30:70 v/v) to yield 6 as white solid powder (3.5 g, 90%); mp: 94-96° C.; ¹H NMR (d₆-DMSO): δ 7.49 (d, J=8.4 Hz, 2H, ArH), 7.41 (d, J=8.4 Hz, 1H, ArH), 7.13 (d, J=8.4 Hz, 2H, ArH), 7.06 (d, J=8.4 Hz, 1H, ArH), 6.45-6.43 (m, 1.5H, ArH), 6.34 (bs, 1.5H, ArH), 5.32-5.29 (m, 0.5H, —CH—), 5.15-5.12 (m, 1H, —CH—), 3.87 (s, 3H, —OCH₃), 3.79 (s, 1.5H, —OCH₃), 3.74 (s, 4.5H, —OCH₃), 2.99-2.94 (m, 4H, 2*-CH₂), 2.80-2.61 (m, 5H, —CH₂), 1.26 (s, 4.5H [CH₃]₃), 1.04 (s, 9H, [CH₃]₃); MS (ESI): m/z 484 [M+Na]⁺. Anal. Calcd (C₂₃H₂₈BrNO₄) C, H, N.

### 6,8-Dimethoxy-1-(2′-methoxybiphenyl-4-ylmethyl)-3,4-dihydro-1H-isoquinoline-2-carboxylic acid tert-butyl ester 7a

A mixture of compound 27 (0.260 g, 0.562 mmol), Palladium(II) acetate (4 mol %), and triphenylphosphine (8 mol %) in anhydrous i-PrOH (15 mL) was stirred under argon conditions at room temperature for 30 min. To this mixture, 2-methoxy phenylboronic acid (0.128 g, 0.84 mmol) and Na₂CO₃ (0.238 g, 2.25 mmol) were added successively. The reaction mixture was refluxed for 16 h, cooled to room temperature, and the solvent was concentrated under reduced pressure. The residue was partitioned between ethyl acetate and saturated NaHCO₃ aqueous solution. Two layers were separated and the aqueous layer was extracted with ethyl acetate. The combined organic layers were washed with water followed by brine and dried over anhydrous Na₂SO₄. The solvents were removed under reduced pressure and the crude residue was purified by flash column chromatography using acetone-hexane (20:80 to 40:60 v/v) to yield 7a as an off-white powder (0.140 g, 51%); 'mp: 126-28° C.; In the NMR spectrum, two sets of peaks were noted, in the ratio of 1:0.3; ¹H NMR (d₆-DMSO): δ 7.40-7.30 (m, 4H, ArH), 7.22-7.20 (m, 3H, ArH), 7.15-7.08 (m, 2H, ArH), 7.04-6.99 (m, 1H, ArH), 6.45 (s, 1H, ArH), 6.43 (s, 0.3H, ArH), 6.35 (s, 1.3H, ArH), 5.41-5.39 (m, 0.3H, —CH), 5.26-5.22 (m, 1H, —CH), 3.89 (s, 3H, —OCH₃), 3.79 (s, 0.9H, —OCH₃), 3.74 (s, 4H, —OCH₃), 3.04-2.95 (m, 2H, —CH₂), 2.86-2.57 (m, 6H, 2*-CH₂), 1.28 (s, 3H, [CH₃]₃), 1.03 (s, 9H, [CH₃]₃); MS (ESI): m/z 512 [M+Na]⁺. Anal. Calcd (C₃₀H₃₅NO₅) C, H, N.

### 6,8-Dimethoxy-1-[4-(4-methoxypyridin-3-yl)benzyl]-3,4-dihydro-1H-isoquinoline-2-carboxylic acid tert-butyl ester 7b

Compound 7b was prepared similarly to 7a using Pd₂(dba)₃ as a catalyst, 2-(dicyclohexylphosphino)2′,4′,6′-tri-i-propyl-1,1′-biphenyl as a ligand, and K₃PO₄ as a base. The crude product was purified by flash column chromatography using acetone-hexane (40:60 to 50:50 v/v) to yield 7b (50%). In the NMR spectrum, two sets of peaks were noted, in the ratio of 1:0.3; ¹H NMR (d₆-DMSO): δ 8.48-8.42 (m, 2.6H, ArH), 7.45-7.37 (m, 2.6H, ArH), 7.30-7.22 (m, 2.6H, ArH), 6.91-6.82 (m, 1.3H, ArH), 6.39 (s, 1H, ArH), 6.31 (s, 1.3H, ArH), 6.26 (s, 0.3H, ArH), 5.67-5.64 (m, 0.3H, —CH), 5.41-5.37 (m, 1H, —CH), 3.92 (s, 3H, —OCH₃), 3.86 (s, 4H, —OCH₃), 3.82 (s, 4H, —OCH₃), 3.76 (s, 1H, —OCH₃), 3.50-3.17 (m, 3H, —CH₂), 2.99-2.55 (m, 4H, 2*-CH₂), 1.35 (s, 3H, [CH₃]₃), 1.13 (s, 9H, [CH₃]₃);); MS (ESI): m/z 513 [M+Na]⁺.

General Procedure for the Synthesis of Hydrochloride Salts 9a/9b

To a solution of t-Boc derivatives 7a/7b in DCM was added trifluoroacetic acid (80 eq) and the reaction mixture stirred for 2 h. The mixture was concentrated, the residue was treated with 37% HCl, filtered, and filtrates were treated with 1N NaOH followed by extraction with chloroform. The chloroform layer was washed with water, dried over Na₂SO₄ and filtered through 3-(diethylenetriamino)propyl functionalized silica gel. The solvents were evaporated under reduced pressure to yield free amine 8a/8b, which was then stirred with 2M HCl in ether to provide hydrochloride salts 9a/9b.

### 4.2.15. 6,8-Dimethoxy-1-(2′-methoxybiphenyl-4-ylmethyl)-1,2,3,4-tetrahydroisoquinoline hydrochloride 9a

The product was obtained as an off-white powder with (80%); mp: 140-42° C.; ¹H NMR (d₆-DMSO): δ 9.31 (s, 1H, —NH₂), 8.57 (s, 1H, —NH₂), 7.47 (d, J=7.8 Hz, 2H, ArH), 7.38-7.27 (m, 4H, ArH), 7.12 (d, J=8.1 Hz, 1H, ArH), 7.06-7.01 (m, 1H, ArH), 6.50 (s, 1H, ArH), 6.45 (s, 1H, ArH), 4.70 (bs, 1H, —CH), 3.76 (s, 6H, 2*OCH₃), 3.73 (s, 3H, OCH₃), 3.18-3.06 (m, 4H, 2*-CH₂), 3.04-2.95 (m, 2H, —CH₂); MS (ESI): m/z 390 [M-(HCl)+H]⁺. Anal. Calcd (C₂₅H₂₈ClNO₃) C, H, N.

### 6,8-Dimethoxy-1-[4-(4-methoxypyridin-3-yl)benzyl]-1,2,3,4-tetrahydroisoquinoline dihydrochloride 9b

The product was obtained as a white powder with 80% yield. ¹H NMR (300 MHz, DMSO-d₆) δ 9.71 (s, 2H, —NH₂), 8.83-8.81 (m, 1H, ArH), 8.69 (s, 1H, ArH), 8.34 (bs, 1H, NH), 7.72-7.70 (m, 1H, ArH), 7.59-7.57 (m, 2H, ArH), 7.43-7.41 (m, 2H, ArH), 6.84-6.45 (m, 2H, ArH), 4.72 (s, 1H, —CH—), 4.09 (s, 3H, —OCH₃), 3.77 (s, 3H, —OCH₃), 3.67 (s, 3H, —OCH₃), 3.26-3.23 (m, 4H, 2*-CH₂), 3.08-3.00 (m, 2H, —CH₂). MS (ES+) m/z 391 [M-(HCl)+H]⁺.

The invention may be further described by means of the following non-limiting examples demonstrating the usefulness of compositions disclosed herein as agents for the destruction of cancer cells and the treatment of cancer.

### EXAMPLES

Screening and Dose Response Assays. 6,6,8-Dimethoxy-1-(2′-methoxybiphenyl-4-ylmethyl)-3,4-dihydro-1H-isoquinoline-2-carboxylic acid tert-butyl ester (7a; EDL-355) was synthesized as described herein in the laboratory of Dr. Duane D. Miller at The University of Tennessee Health Science Center. Briefly, the primary cultures of astrocytes and cultures of C6 glioma cell lines were handled identically with respect to treatment concentrations and manipulations of cells for the screening assays. The cells were trypsinized and transferred to 96-well plates at a cell density of 10³ cells/mm² in the wells. The cells were grown overnight in 100 μL of 10% FCS BME in a 37° C. incubator containing a humid, 5% CO₂ atmosphere.

EDL-355 was dissolved completely to make a 100 μM stock solution and diluted to produce a series of concentrations.

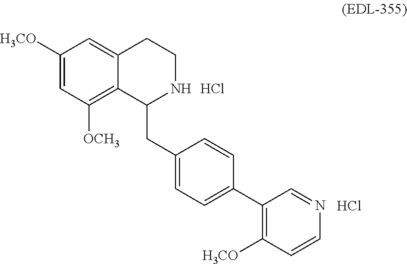

A 20 μL aliquot of these initial solutions was added to 180 μl of 2% FCS BME to produce the test concentration. The vehicle solution was tested as a control. Dilutions were performed such that co-solvent concentrations did not vary for a particular experiment. Immediately before treatment, the 10% FCS BME was removed from the cells and replaced with the 180 μA of the treatment medium. The cultured cells (normal primary cultures of rat brain astrocytes or C6 glioma purchased from ATCC) were incubated with test compound for 4 days. The cells were fixed with 4% paraformaldehyde, stained with 0.1% Cresylecth violet stain, and quantitated. The screening data was collected as four wells for each dose per compound (screening) or concentration (dose response curve). Also, the average growth of 8 wells with no treatment was used as a negative control for each plate (100% growth). The cells for dose response curves were grown in the same media and were handled in a similar manner as in the screening assays.

Data Analysis. The cytotoxic character of each compound was reported as the percent survival, calculated as the average A₅₆₀ for treated cells divided by A₅₆₀ of untreated (100% control) cells, and expressed as a percentage. Values less than 100% indicate a cytostatic or cytotoxic effect. Dose response curves and EC50 values were attained via plots of percent survival vs. concentration.

### Chiral Separation of THI-Analogs

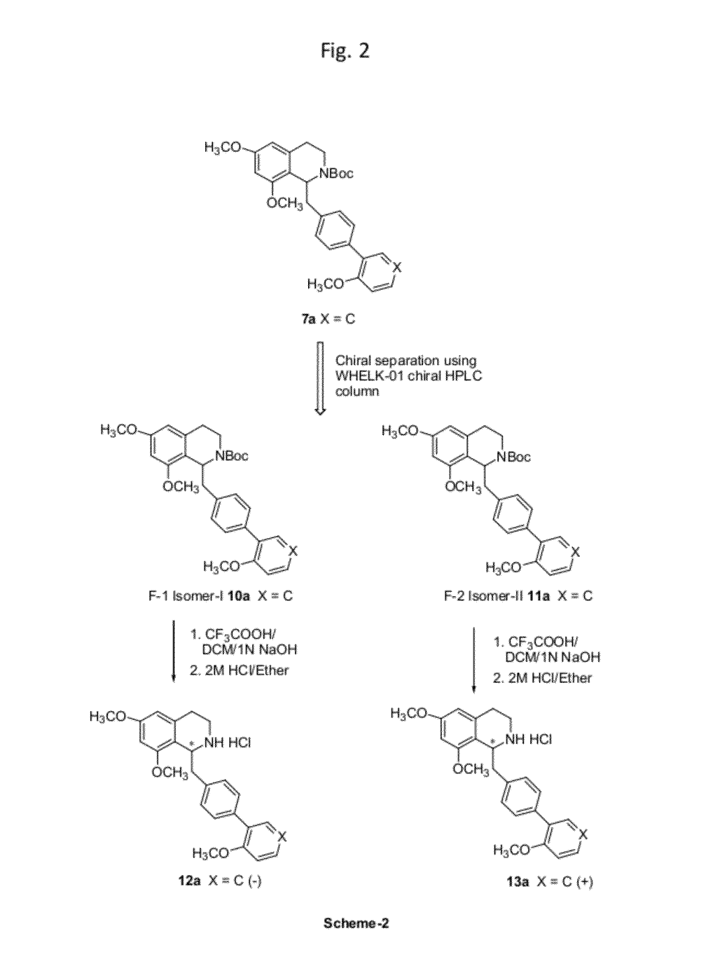

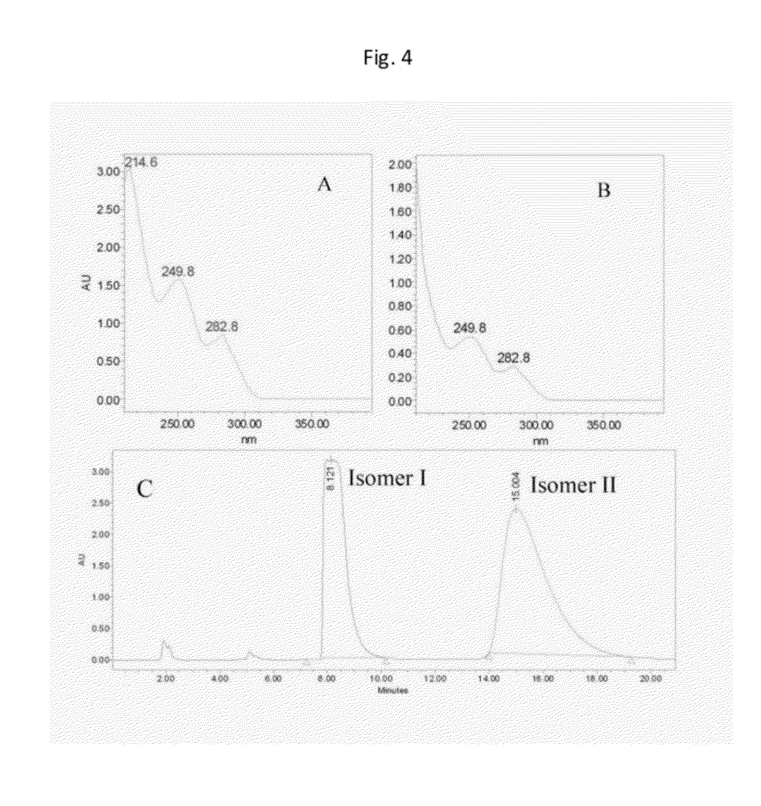

Enantiomers of chiral drugs often show significant differences in their pharmacokinetics (PK), pharmacodynamics (PD), and adverse reactions. Therefore, with the aim of elucidating an enantiopharmacological profile of compound 9a, the inventors performed the chiral resolution of its precursor N-substituted THI analog 7a by HPLC method. The inventors resolved the individual isomers by (R, R) WHELK-01 chiral HPLC column (Regis technologies, Morton Grove, Ill.), which enabled them to directly and efficiently obtain both enantiomers in nearly 100% optical purity with an 85:15 ratio of Hexane and isopropanol (FIG. 2). The first fraction eluted at 8.12 min and second fraction eluted at 15.00 min. The collected fractions from each isomer were pooled and evaporated under vacuum to give the residues as a first fraction (Isomer-I: (10a)) and a second fraction (Isomer-II: (11a)). The purity of Isomer-I (10a) was 100% and that of Isomer-II (11a) was 99% (FIG. 4). After resolution, both isomers (10a & 11a) were treated with trifluoroacetic acid in dichloromethane, followed by reaction with 2M HCl in ether to yield corresponding hydrochloride salts 12a and 13a, respectively (FIG. 2, Scheme-2). The inventors examined the specific rotations of 12a and 13a in methanol solution using DigiPol 781 (Rudolph Instruments, Denville, N.J.) automatic polarimeter and their specific rotations were [α]_{D}=−35.0 and [α]_{D}=+40.2 (t=25° C.), respectively. Biological studies revealed that (+) 13a was the most active, being nearly 21 fold more potent than (−) 12a on rat C6 glioma cell lines [(+) 13a: 0.26 μM; (−) 12a: 5.39 μM]. This confirmed that the significant anitiglioma activity of (R, S)-7a (0.63 μM) was primarily due to (+) 13a (see Table 1).

TABLE 1

| | Molecular | Molecular | | EC50 μM | |
|---|---|---|---|---|---|
| STRUCTURE | Formula | Weight | Log P | Astrocyte | EC50 μM C6 |
| 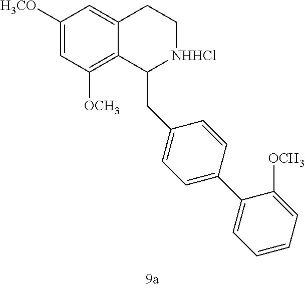 | C₂₅H₂₈ClNO₃ | 425.95 | 4.87 | 10.15 8.515 | 0.5004 0.182 |
| 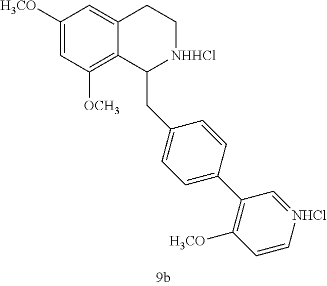 | C₂₄H₂₈Cl₂N₂O₃ | 463.40 | 3.53 | 13.7 | 0.08 |
| 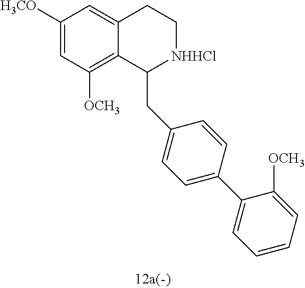 | C₂₅H₂₈ClNO₃ | 425.95 | 4.87 | 15.4 | 5.4 |
| 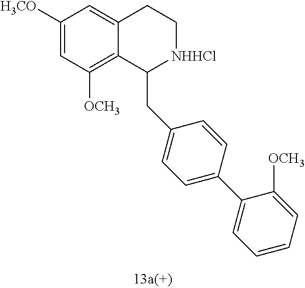 | C₂₅H₂₈ClNO₃ | 425.95 | 4.87 | 13.3 | 0.3 |

What is claimed is:

1. A compound of Formula (I)
where R¹ is 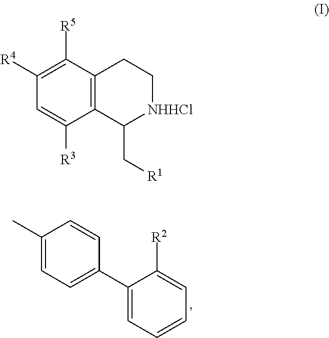 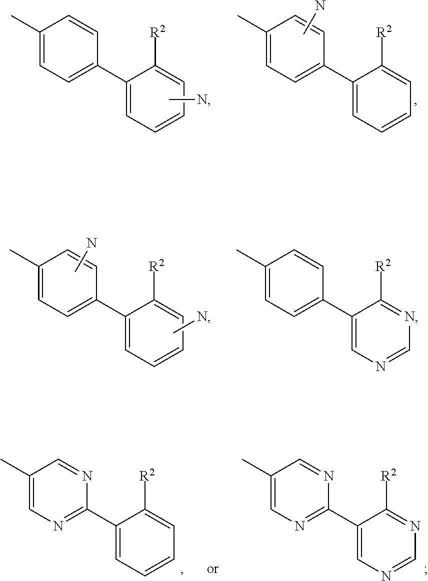
R² is —OCH₃, —CF₃, —NHSO₂CH₃, —NHCOCH₃, —SO₂CH₃, —N(CH₃)₂, —CN, —NO₂, —NH₂, —CO₂CH₃, —OCF₃, —CH₃, —F, —Br, or —I;
R³ is —OCH₃; and
R⁴ and R⁵ are —H or —OCH₃, wherein when R⁴ is —H, R⁵ is —OCH₃ and when R⁴ is —OCH₃, R⁵ is —H.

2. The compound of claim 1 wherein R¹ is

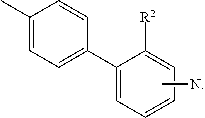

3. The compound of claim 1 wherein R² is —OCH₃.

4. The compound of claim 1 wherein R¹ is 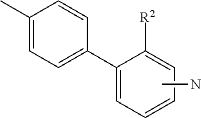
and R² is —OCH₃.

5. A method comprising administering a compound of Formula (I) to a patient to kill cancer cells, reduce tumor size, and treat cancer, 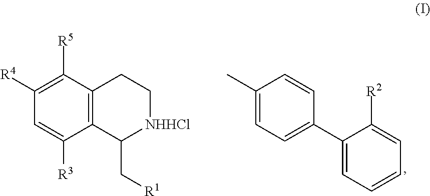
wherein R¹ is 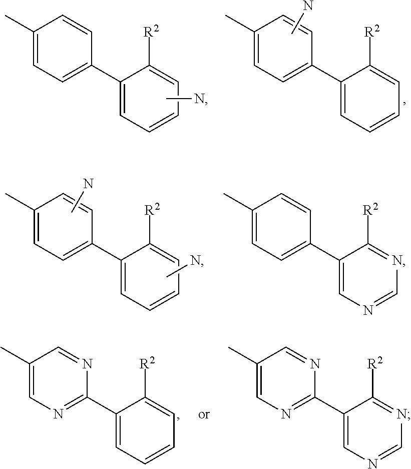
R² is —OCH₃, —CF₃, —NHSO₂CH₃, —NHCOCH₃, —SO₂CH₃, —N(CH₃)₂, —CN, —NO₂, —NH₂, —CO₂CH₃, —OCF₃, —CH₃, —F, —Br, or —I;
R³ is —OCH₃; and
R⁴ and R⁵ are —H or —OCH₃, wherein when R⁴ is —H, R⁵ is —OCH₃ and when R⁴ is —OCH₃, R⁵ is —H.

6. The method of claim 5 wherein the compound is administered to kill cancer cells.

7. The method of claim 5 wherein the compound is administered to reduce tumor size.

8. The method of claim 6 wherein the cancer cells are glioblastoma cells.

9. The method of claim 6 wherein R¹ is 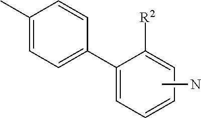
and R² is —OCH₃.

10. The method of claim 8 wherein R¹ is 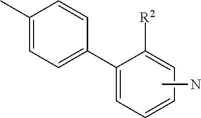
and R² is —OCH₃.
